(19) United States
(12) Patent Application Publication
(10) Pub. No.: US 2016/0317418 A1
(43) Pub. Date: Nov. 3, 2016

(54) METHOD OF IMPROVING THE APPEARANCE OF SKIN AND COMPOSITIONS THEREFOR

(71) Applicant: The Procter & Gamble Company, Cincinnati, OH (US)

(72) Inventors: Tomohiro HAKAZAKI, Cincinnati, OH (US); Leo Timothy LAUGHLIN, II, Mason, OH (US); John Erich OBLONG, Loveland, OH (US)

(21) Appl. No.: 15/141,976

(22) Filed: Apr. 29, 2016

Related U.S. Application Data
(60) Provisional application No. 62/155,712, filed on May 1, 2015.
(60) Provisional application No. 62/162,054, filed on May 15, 2015.

(51) Int. Cl. A61K 8/60; A61K 8/49; A61K 8/02; A61Q 19/02; A61K 8/06

(52) U.S. Cl. CPC A61K 8/602; A61Q 19/02; A61K 8/06; A61K 8/025; A61K 8/022; A61K 8/4926; A61K 2800/592

(57) ABSTRACT

A cosmetic method for reducing the level of High-Mobility Group Protein B1 released from keratinocytes. The method includes identifying a target portion of skin where reduction of HMGB1 level is desired, and topically applying a nicotinamide riboside containing cosmetic composition to the target portion of skin during a treatment period, which is sufficient to reduce the level of HMGB1 released by keratinocytes. By reducing HMGB1 protein level, melanocyte dendricity can be reduced, thereby resulting in less melanin being taken into keratinocytes. The reduction in melanin transfer may provide a way to treat skin pigmentation disorders such as hyperpigmented spots.

### FIELD

The present disclosure is directed generally to methods and compositions for lightening skin. More specifically, the present disclosure is directed to methods and compositions that include nicotinamide riboside for providing a skin lightening benefit.

### BACKGROUND

Skin pigment irregularities such as skin tone evenness and/or the appearance of hyperpigmented spots are common across ethnic and racial groups and are commonly perceived as cosmetic blemishes. Skin pigmentation and the broader cosmetic concept of skin tone are highly complex conditions with multiple and overlapping etiologies. Disorders of skin pigment production and distribution can occur as a function of intensity and duration of UV radiation exposure, life style habits, chronological age, endocrine functioning, and disease state and are found ubiquitously in older populations. Since these conditions can manifest as a function of individual predisposition, they can pose a significant treatment challenge. As such, cosmetic compositions and methods for addressing these consumer concerns are continuing areas of high interest.

The color of normal human skin is due primarily to varying amounts and distribution of melanin, hemoglobin, and carotenoids. Of these pigments, melanin, and eumelanin in particular, is of primary significance to cosmetic skin treatment protocols. Eumelanin is responsible for imparting a brown or black tone to skin, while pheomelanin imparts a red to pink hue. Melanin is produced by specialized cells in the skin called melanocytes via a complex series of chemical and enzymatic reactions, mainly involving the copper containing enzyme tyrosinase. Once synthesized, the melanin granules are packaged into melanosomes and transferred along cellular dendrites to the surrounding keratinocytes, the most abundant cell type in the epidermis. Since there are approximately 36 keratinocytes for each melanocyte in the epidermis, the melanocytes must rely on their dendritic structures to “reach” neighboring keratinocytes for melanosome transfer. Melanosome carrying keratinocytes then migrate upward toward the skin surface, thereby darkening the skin.

The biochemical pathway for eumelanin production is relatively well elucidated. Summarily, eumelanin forms through a series of oxidative reactions involving the amino acid tyrosine in the presence of the enzyme tyrosinase. Tyrosinase converts tyrosine to dihydroxyphenylalanine (DOPA) and then to dopaquinone. Subsequently, dopaquinone is converted to dopachrome through auto-oxidation, and finally to dihydroxyindole or dihydroxyindole-2-carboxylic acid (DHICA), which polymerize to form eumelanin. However, there are many regulatory elements involved in cell signaling, in the transport of melanosomes within the melanocyte, and in the transfer of melanosomes to the keratinocytes. Some pathways are relatively well elucidated, while others are not. Cosmetic agents that affect one or more of these pathways can be useful for lightening skin, and particularly for improving the appearance of hyperpigmented skin and/or other skin pigmentation disorders.

Vitamin B₃ compounds such as niacin and its derivatives are known for their use as skin lightening agents. U.S. Pat. No. 4,096,240 (“Mathur”) refers to niacin as effective in skin lightening. This material is postulated to operate by retarding melanin dispersion or distribution into the epidermis. Since unpleasant skin flushing occurs with niacin, Mathur discloses the use of niacinamide, which is sometimes referred to as nicotinamide, as a substitute. Compositions based upon niacinamide may be effective, but there is still a need to identify other suitable skin care actives, which can be used to formulate cosmetic compositions that provide improved skin lightening benefits.

U.S. Pat. No. 8,106,184 (“Sauve”) is directed to compositions and methods that use nicotinoyl ribosides and nicotinamide riboside for increasing intracellular levels of nicotinamide adenine dinucleotide (“NAD+”) in cells and tissues. Sauve discloses that skin can be protected from aging (e.g., developing wrinkles, loss of elasticity, etc.) by treating skin or epithelial cells with a nicotinoyl riboside or derivative compound that increases the level intracellular NAD+. Sauve also discloses that exemplary skin afflictions or skin conditions treatable by its methods include disorders or diseases associated with or caused by inflammation, sun damage or natural aging. However, Sauve only discusses the effect of nicotinoyl ribosides on the NAD+ metabolic pathway to the exclusion of other biological pathways. Not all biological pathways related to skin pigmentation are well elucidated, and Sauve does not disclose how modulating the NAD+ pathway can improve the appearance of hyperpigmented skin. Thus, there is still a need to explore other biological pathways related to skin pigmentation in order to provide improved methods of treating these disorders.

U.S. Publication No. 2005/0267023 (“Sinclair”) is directed to methods and compositions for modulating the life span of a cell or its resistance to stress, comprising modulating the flux through the NAD+ salvage pathway in the cell, which may comprise increasing the level or activity of a protein selected from the group consisting of NPT1, PNC1, NMA1 and NMA2. Sinclair discloses an embodiment wherein the amount and/or activity of nicotinamide is reduced. Sinclair also discloses an embodiment wherein the NAD+ salvage pathway in a cell is stimulated by contacting the cell with nicotinamide riboside or a biologically active analog and/or prodrug thereof. Increasing the lifespan of a cell or its resistance to stress is important for the overall health of the cell and/or tissue, but there still remains a need to identify effective methods of treating skin pigmentation disorders for people in need of such treatment.

PCT Publication No. WO 2014/163896 (“Shah”) is directed to a cosmetic composition comprising 3,3′ thiodipropionic acid (“TDPA”) in combination with nicotinamide (a.k.a. niacinamide), which allegedly provide an additive or synergistic skin lightening benefit. According to Shah, TDPA inhibits tyrosinase activity thereby inhibiting melanogenesis, while nicotinamide inhibits melanosome uptake from the melanocyte into the keratinocyte. However, there is still a need to identify the specific biological pathways that directly impact skin pigmentation as well as cosmetic agents that provide skin lightening benefits via these pathways.

PCT Pub. No. WO 2015/066382 (“Deren-Lewis”) relates to methods of using nicotinamide riboside to promote the increase of intracellular levels of nicotinamide adenine dinucleotide (NAD+) in cells and tissues for improving cell and tissue survival. Deren-Lewis discloses the use of topical nicotinamide riboside compositions for treating a variety of skin conditions by modulating the NAD+ pathway. However, there is still a need to elucidate the biological pathways that impact skin pigmentation as well as cosmetic agents that provide skin lightening benefits via these pathways.

Accordingly, it would be desirable to provide methods and compositions that inhibit HMGB1 level and/or activity. It would also be desirable to provide compositions comprising an effective amount of nicotinamide riboside, which inhibits HMGB1 level and/or activity, and methods of using such compositions to provide a skin lightening benefit.

### SUMMARY

A cosmetic method for reducing the level of HMGB1 released from keratinocytes is provided. In one aspect, the method is directed to identifying a target portion of skin where reduction of HMGB1 level is desired and/or needed, and topically applying a cosmetic composition to the target portion of skin, wherein the cosmetic composition comprises an effective amount of nicotinamide riboside and the treatment period is sufficient to reduce the level of HMGB1 released by keratinocytes. In another aspect, the method is directed to lightening skin by topically applying a cosmetic composition to a target portion of skin during a treatment period, wherein the cosmetic composition is formulated with about 0.005% to about 20% by weight nicotinamide riboside and the treatment period is sufficient to lighten the target skin portion. In still another aspect, the method is directed to improving the appearance of a hyperpigmented spot by topically applying a cosmetic composition to at least a portion of the hyperpigmented spot during a treatment period, wherein the cosmetic composition is formulated with about 0.005% to about 20% by weight nicotinamide riboside and the treatment period is sufficient to reduce the size of the hyperpigmented spot.

### BRIEF DESCRIPTION OF THE DRAWINGS

FIG. 1 is a chart showing the amount of HMGB1 released from keratinocytes exposed to UVB.

FIG. 2 shows an image of a face with a portion of the cheek masked.

### DETAILED DESCRIPTION

It is known that niacinamide and nicotinamide riboside, which are both NAD+ precursors, can be used to modulate the NAD+ pathway to treat cells for some of the symptoms commonly associated with aging. It is also known that niacinamide inhibits the uptake of melanosomes by keratinocytes from melanocytes. But it was unknown whether nicotinamide riboside could provide a similar benefit since it is not clear whether the NAD+ pathway impacts the uptake of melanosomes from melanocytes into keratinocytes. Surprisingly, when nicotinamide riboside was tested for its ability to inhibit the uptake of melanosomes from melanocytes into keratinocytes, no significant effect was observed, which suggests that nicotinamide riboside may not be suitable for use as a skin tone agent. However, through further research, it has now been discovered that nicotinamide riboside reduces the amount of HMGB1 protein released from keratinocytes in acute stress models, and thus may be suitable for use as a topical skin tone agent.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages are by weight of the corresponding composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### DEFINITIONS

“Apply” or “application”, as used in reference to a composition, means to apply or spread the compositions of the present invention onto a human skin surface.

“Cosmetic” means providing a desired visual effect on an area of the human body. The visual cosmetic effect may be temporary, semi-permanent, or permanent.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives. The compositions herein may optionally include one or more cosmetic agents in addition to an effective amount of nicotinamide riboside. In some embodiments, cosmetic agents may be incorporated in a cosmetic composition comprising a dermatologically acceptable carrier suitable for topical application to skin.

“Dendricity” means the total length of dendrites measured on one or more melanocytes. Dendricity may be measured with an Incucyte ZOOM® live cell imaging system available from Essen Bioscience, Ann Arbor, Mich. “Reduced dendricity” means that the total length of the dendrites is reduced. A suitable method of determining melanocyte dendricity is disclosed in U.S. Ser. No. 14/847,036 filed by Hakozaki et al., on Sep. 8, 2015 and titled “Compositions and Methods for Inhibiting HMGB1 Activation of Melanocytes.”

“Dendrite” means a branched, tendril-like projection of a melanocyte that acts to transfer melanosomes from the melanocyte cell body to adjacent keratinocytes.

“Disposed” means an element is positioned in a particular place relative to another element.

“Effective amount” means an amount of nicotinamide riboside sufficient to provide a skin lightening benefit (e.g., improve the appearance of a hyperpigmented spot) over the course of a treatment period.

“Hyperpigmented” and “hyperpigmented spot” mean a localized portion of skin with relatively high melanin content. Examples of hyperpigmented skin include, but are not limited to age spots, melasma, chloasma, freckles, post inflammatory hyperpigmentation, sun-induced pigmented blemishes and the like.

“Improve the appearance of” means providing a measurable, desirable change or benefit in skin tone appearance and/or the appearance of a hyperpigmented spot, which may be quantified by a reduction in the Spot Area Fraction and/or an increase in L* value. Exemplary methods for determining these values are described in more detail below.

“L*a*b*” refers to the commonly recognized color space specified by the International Commission on Illumination (“CIE”). The three coordinates represent (i) the lightness of the color (i.e., L*=0 yields black and L*=100 indicates diffuse white), (ii) the position of the color between magenta and green (i.e., negative a* values indicate green while positive a* values indicate magenta) and (iii) the position of the color between yellow and blue (i.e., negative b* values indicate blue and positive b* values indicate yellow).

“Safe and effective amount” means an effective amount of nicotinamide riboside that is low enough to avoid serious side effects (within the scope of sound medical judgment).

“Skin tone” means the overall appearance of melanin in the skin caused by the systemic, rather than transient, synthesis of melanin. Skin tone is typically characterized over a relatively large area of the body. (e.g., the face, arm, chest, shoulder, abdomen or a substantial portion of one or more of these). An exemplary area for evaluating skin tone is about 100 mm² or more. Skin tone may be determined using a suitable image analysis technique. For example, overall lightness can be determined by using the L* coordinate in the L*a*b* color space (International Commission on Illumination). Chromophore mapping such as melanin mapping and melanin concentration may also be used as an indicator of overall skin tone.

“Skin tone agent” means a cosmetic agent intended to be applied to the skin for the purpose of effectuating a change in skin tone and/or skin pigmentation.

“Skin lightening” means one or more of the following: overall lightening of skin tone, reduction in spot area and/or lightening of hyperpigmented regions, including age spots, melasma, chloasma, freckles, post inflammatory hyperpigmentation or sun-induced pigmented blemishes. Changes in skin lightening may be determined by visual grading and/or by measuring a change in L* value in a region of interest, for example, using a spectrophotometer or the like.

“Treatment period,” as used herein means the length of time and/or frequency that a material or composition is applied to a target skin surface.

### Cosmetic Compositions

Various cosmetic compositions, and more specifically cosmetic compositions for topical application to skin, are provided. These cosmetic compositions comprise a safe and effective amount of nicotinamide riboside. The cosmetic compositions herein may be provided in various product forms that include, but are not limited to, solutions, suspensions, lotions, creams, gels, toners, sticks, sprays, aerosols, ointments, cleansing liquid washes and solid bars, pastes, foams, mousses, shaving creams, wipes, strips, patches, electrically-powered patches, hydrogels, film-forming products, facial and skin masks (with and without insoluble sheet), make-up such as foundations, eye liners, and eye shadows, and the like. The cosmetic composition form may follow from the particular dermatologically acceptable carrier chosen, if present in the composition. Cosmetic compositions herein may be made using conventional methods for making cosmetic compositions. The cosmetic composition form may follow from the particular dermatologically acceptable carrier chosen, if present in the composition.

The cosmetic compositions herein may be made using conventional methods of making such compositions. For example, a cosmetic composition comprising an effective amount of nicotinamide riboside may be made by mixing nicotinamide riboside with a dermatologically acceptable carrier at an amount of from 0.05%, 0.5%, 1%, 2%, 3%, 4% or 5% to 20%, 15%, 10%, 8% or 6% by weight of the composition. It is known that nicotinamide riboside can undergo hydrolysis in an aqueous environment, depending on temperature and time in solution. This can be especially problematic for cosmetic compositions, which may be stored (e.g., in a warehouse, during shipping or on a store shelf) for a relatively long period of time (e.g., from 1 to 3 weeks or even from 1 to 6 months) at variety of temperatures. Typical storage temperatures for cosmetic compositions may range from 5° C. to 45° C. Thus, in order to help ensure an effective amount of nicotinamide riboside is provided in an aqueous skin-care composition (i.e., a composition that includes water) at the time of use, the amount of nicotinamide riboside added to the composition during manufacture should be adjusted according to the known chemical kinetics of nicotinamide riboside and water. A discussion of the reaction kinetics of nicotinamide riboside and water can be found in a publication by Ferraz, et al., titled “Kinetic α-Deuterium Isotope Effects for Enzymatic and Nonenzymatic Hydrolysis of Nicotinamide-β-Riboside,” Archives of Biochemistry and Biophysics Vol. 191, No. 2, December, pp. 431-436, 1978.

### Nicotinamide Riboside

The methods and topical cosmetic compositions herein include a safe and effective amount of nicotinamide riboside. Nicotinamide riboside (CAS No. 1341-23-7) has the formula:

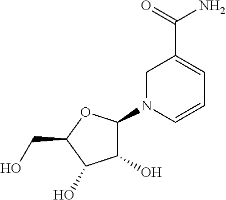 [SMILES: NC(=O)C1=CC=CN([C@@H]2O[C@H](CO)[C@@H](O)[C@H]2O)C1]

Some examples of nicotinamide riboside and their methods of manufacture are described in U.S. Pat. No. 8,106,184. As used herein, the term “nicotinamide riboside” includes salts of nicotinamide riboside (e.g., nicotinamide riboside chloride).

The cosmetic compositions herein may include from 0.05%, 0.5%, 1%, 2%, 3%, 4% or 5% to about 20%, 15%, 10%, 8% or 6% by weight of the cosmetic composition of nicotinamide riboside. It is to be appreciated that the amount of nicotinamide riboside in the present compositions may vary depending on how much HMGB1 reduction is desired. The foregoing amounts may be added at the time of manufacture and/or present at the time of use, depending on the desired level of benefit desired. For example, the amount of nicotinamide riboside added to the composition at the time of manufacture and/or present at the time of use may be sufficient to reduce HMGB1 by at least 10%, 15%, 20%, 25%, 30%, 35%, 40%, 45%, 50%, 55%, 60%, 65%, 70%, 75%, 80%, 85%, 90%, 95% or even 100%. It may be particularly desirable for the safe and effective amount of nicotinamide riboside to reduce HMGB1 protein levels and/or activity in stressed keratinocytes (i.e., keratinocytes exposed to a stressor such as ultraviolet radiation) to pre-stress levels or below, which could result in a reduction of greater than 100%. HMGB1 level can be determined by using a conventional HMGB1 ELISA kit (e.g., the High Mobility Group Box 1 Protein (HMGB1) ELISA Kit available from IBL International as REF # ST51011) according to the manufacturer's instructions.

Reducing HMGB1 levels, and thus dendrite stimulation caused by HMGB1 may improve the appearance of hyperpigmented spots and/or overall skin tone. In some instances, the improvement may correspond to a positive change in L* value (i.e., a ΔL* value that is greater than 0, but typically less than 100) when the nicotinamide riboside is applied during a treatment period. In some instances, the ΔL* value may be from 0.1 to 10, from 0.2 to 5, or even from 0.3 to 3. Additionally or alternatively, the improvement in appearance may correspond to a reduction in Spot Area Fraction of at least 2% (e.g., from 2% to 100%, from 5% to 70%, from 10% to 40%, from 15% to 25%).

It is to be appreciated that reducing HMGB1 level is not considered “inhibiting melanin uptake” as that term is used when referring to the mechanism of action believed to be associated with niacinamide. In some instances, it may be desirable to select an effective amount of nicotinamide riboside that does not inhibit (or promote) melanin uptake. In this way, it may be possible to provide improved skin appearance benefits by formulating compositions that include skin tone agents that function via different biological pathways (e.g., niacinamide and nicotinamide riboside).

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In certain embodiments, the dermatologically acceptable carrier is in the form of an emulsion. Emulsions may be generally classified as having a continuous aqueous phase (e.g., oil-in-water and water-in-oil-in-water) or a continuous oil phase (e.g., water-in-oil or oil-in-water). The oil phase of the present invention may comprise silicone oils, non-silicone oils such as hydrocarbon oils, esters, ethers, and the like, and mixtures thereof. The aqueous phase typically comprises water and water-soluble ingredients (e.g., water-soluble moisturizing agents, conditioning agents, anti-microbials, humectants and/or other skin care actives). However, in some instances, the aqueous phase may comprise components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). Emulsions may also contain an emulsifier, e.g., from about 1% to about 10% or from about 2% to about 5% based on the weight of the carrier. Emulsifiers may be nonionic, anionic or cationic. Some non-limiting examples of emulsifiers are disclosed in U.S. Pat. No. 3,755,560 to Dickert et at; U.S. Pat. No. 4,421,769 to Dixon et al.; and McCutcheon's Detergents and Emulsifiers. North American Edition, pages 317-324 (1986).

The carrier may contain one or more dermatologically acceptable, hydrophilic diluents. As used herein, “diluent” includes materials in which the nicotinamide riboside can be dispersed, dissolved, or otherwise incorporated. Hydrophilic diluents include water, organic hydrophilic diluents such as lower monovalent alcohols (e.g., C1-C4) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Optional Ingredients.

The present compositions may optionally include one or more additional ingredients commonly used in cosmetic compositions (e.g., colorants, skin tone agents, skin anti-aging agents, anti-inflammatory agents, sunscreen agents, combinations of these and the like), provided that the additional ingredients do not undesirably alter the skin lightening benefit provided by the composition. In some instances, it may be desirable to select skin tone agents that function via different biological pathways so that the actives do not interfere with one another, which could otherwise reduce the efficacy of both agents. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition. The additional ingredients, when incorporated into the composition, should be suitable for use in contact with human skin tissue without undue toxicity, incompatibility, instability, allergic response, and the like. Some nonlimiting examples of additional ingredients which may be suitable for use herein are described in U.S. Publication Nos. 2002/0022040; 2003/0049212; 2004/0175347; 2006/0275237; 2007/0196344; 2008/0181956; 2010/00092408; 2008/0206373; 2010/0239510; 2010/0189669; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533. Some further nonlimiting examples of additional ingredients, which may be particularly suitable for use in the present compositions, are provided below.

In some instances, the compositions herein can include from 0.001% to 40% (e.g., from 1% to 30%, or from 2% to 20%) of one or more particulate materials and/or cosmetic powders to provide acute look and/or feel benefits. These particulates can, for instance, be platelet shaped, spherical, elongated or needle-shaped, or irregularly shaped; surface coated or uncoated (e.g., hydrophobically coated); porous or non-porous; charged or uncharged; and can be added to the current compositions as a powder or as a pre-dispersion. For example, pigmentary-grade metal oxide particles (e.g., having an average primary particle size greater than 100 nm or from 100 nm to 500 nm) may optionally be included to provide an appearance benefit. Some nonlimiting examples of particulate materials for use herein are described in U.S. Publications Nos. 2012/0021027, 2010/0074928, 2010/0003205, 2010/0003293 and 2013/0243835.

In another example, the compositions used in accordance with the present method may include powders in the form of spherical particles, which provide an acute look and/or feel benefit. Spherical particle powders tend to improve the speed that the product appears to absorb into the skin, which helps provide increased control over product application. Spherical particle powders herein have a median particle size of from 2 μm to 40 μm, (e.g., from 3 μm to 25 μm or even from 5 μm to 15 μm). Spherical particles powders can also increase the smooth feeling of the product film on the skin. Accordingly, it may be desirable to select spherical particles that have no tackiness and a rubber hardness (as measured by Durometer A defined in JIS K 6253) in the range of 10 to 90, (e.g., 20 to 80 or even from 25 to 75). In a particularly suitable example, the composition includes 2% to 20% (e.g., 4% to 12%) spherical silicone elastomer particles or spherical starch particles. The amount of silicone elastomer powder in the composition is determined based on the particulate material being in neat form (i.e., not swollen in solvent). Some nonlimiting examples of spherical particle powders are described in co-pending U.S. Ser. Nos. 14/596,360 and 14/596,374, filed by Jansen, et al., on Jan. 14, 2015.

### Methods of Use

The methods herein include identifying a target portion of skin (e.g., a facial skin surface such as the forehead, perioral, chin, periorbital, nose, and/or cheek) in need of treatment and/or where treatment is desired and applying a safe and effective amount of nicotinamide riboside to the target portion of skin. The nicotinamide riboside may be incorporated into a suitable cosmetic composition using conventional methods for making cosmetic compositions. Without intending to be bound by theory, it is believed that application of an effective amount of nicotinamide riboside to skin reduces HMGB1 level and/or activity, and thus reduces melanocyte dendricity. In this way, a skin-lightening benefit may be provided to the target portion of skin. In some instances, the target portion of skin may not exhibit a skin pigmentation disorder, but a user (e.g., a relatively young user) may still wish to target such an area of skin if it is one that typically develops a skin pigmentation disorder later in life (e.g., skin surfaces that are typically not covered by clothing, such as facial skin surfaces, hand and arm skin surfaces, foot and leg skin surfaces, and neck and chest skin surfaces). In this way, the present methods and compositions may be used as a preventative measure. In some instances, the level and/or activity of HMGB1 present in the target portion of skin may be measured and compared to a reference amount to determine if treatment is needed and/or desired. Cosmetic compositions containing an effective amount of nicotinamide riboside may be applied to the target skin portion and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed.

The treatment period is ideally of sufficient time for the nicotinamide riboside containing composition to improve the appearance of the target portion of skin, which may correspond to a reduction in the size of hyperpigmented spot and/or an increase in lightness. The treatment period may last for at least 1 week (e.g., about 2 weeks, 4 weeks, 8 weeks, or even 12 weeks). In some instances, the treatment period will extend over multiple months (i.e., 3-12 months) or multiple years. In some instances, a cosmetic composition containing an effective amount of nicotinamide riboside may be applied most days of the week (e.g., at least 4, 5 or 6 days a week), at least once a day or even twice a day during a treatment period of at least 2 weeks, 4 weeks, 8 weeks, or 12 weeks.

The cosmetic compositions herein may be applied locally or generally. In reference to application of the composition, the terms “localized”, “local”, or “locally” mean that the composition is delivered to the targeted area (e.g., a hyperpigmented spot or portion thereof) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied more generally or broadly to one or more skin surfaces. In certain embodiments, the compositions herein may be used as part of a multi-step beauty regimen, wherein the present composition may be applied before and/or after one or more other compositions.

### Test Methods

### Imaging Method

This method provides a means for capturing a reproducible and analyzable image for determining L*a*b* values and Spot Area Fraction. It is to be appreciated that any suitable image capture device along with imaging software and other associated ancillary equipment (e.g., computer and lights) which are equivalent to those described in this method may be used. The imaging system in this method incorporates a FUJI-S2 Pro brand CCD SLR digital camera which delivers a 6 megapixel uncompressed image (BMP) and a raw image file (RAF). Prior to taking a photograph, the test subject is illuminated with a JTL 1000W flash through two linear polarizers in crossed axis orientation. A chart containing Munsell Color Standard Neutral N2-N9.5 are captured in every image for standardization and color correction purposes.

In preparation for image capture, test subjects are required to wash their faces and wait for at least 15 minutes to let their face dry. The hair of the subject is covered with a hairnet and the head and shoulders of the subject are covered with a black cloth. All jewelry that can be seen in an image area of interest is removed. The subjects are equilibrated in a control room at 20-25° C. and 40-60% relative humidity for 30 minutes. Next, each subject is suitably positioned, in front of the camera and one or more images of each side of the face are captured. The captured image(s) are then processed by converting the raw image to a .jpg file format.

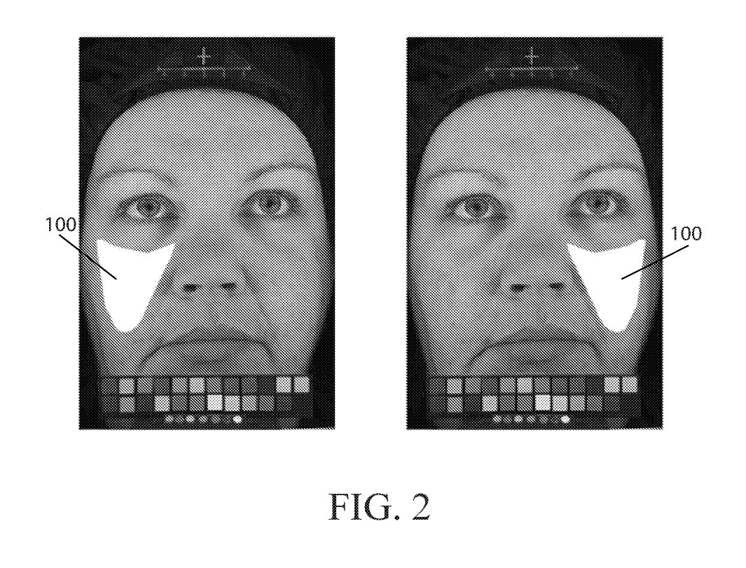

Next, the .jpg format image is analyzed by a computer with suitable image analysis software. In some instances, it may be desirable to analyze only a portion of the image (i.e., a region of interest (“ROI”)). The ROI may be “masked,” for example, as shown in FIG. 2, using image editing software such as Photoshop® or Image J® brand software. The masked region (e.g., cheek 100 in FIG. 2) can then be isolated and analyzed as a separate image. It is to be appreciated that the image need not necessarily be masked for suitable analysis, and in some instances the entire image may be analyzed. In some instances, it may be desirable to reduce the size of the image, mask and/or region of interest by several pixels (e.g., between 5 and 15 pixels) around the outer edge of the image where some shadowing may occur.

Since color may be perceived as being relative, depending on, for example, which instruments and/or imaging system is used, it can be important to color correct the image or region of interest for each subject using a suitable color correction technique (e.g., according to International Color Consortium standards and practices), which helps make the color determination by the system less instrument specific. The RGB values in the captured images, which are device dependent, are converted to L*a*b* values. The L*a*b* values can be calculated using a suitable RGB conversion tool (e.g., software installed on the computer or a suitable conversion tool found online) The conversion from RGB values to L*a*b* values can be performed on the entire image, a ROI or on one or more individual pixels. The resulting L*a*b* values may be averaged to provide average values for the image or a region of interest.

Spot Area is the total area of spots (in pixels) detected in the desired ROI. Spots are detected by comparison of localized detection of lower gray density objects from higher gray density background in the desired channel of the RGB color space. The detected objects are further classified by shape and size.

Spot Area Fraction may be represented by the equation below.

$$S A F ( \% ) = \frac{Cumulative spot area within the ROI * 100}{Area of the ROI}$$

The change in SAF (“ΔSAF”) is the difference between the spot area (normalized to the ROI) after a treatment period and the spot area (normalized to the ROI) just prior to treatment (e.g., SAF_{final}−SAF_{baseline}). Since seasonal changes may affect results, the relative change to a control population (“ΔΔSAF”) is useful and is the difference between the ΔSAF for the treatment period and a control population for the same period (e.g., ΔSAF_{test composition}−ΔSAF_{vehicle} control). A lower percentage reflects a reduction in spot area. ΔSAF for a treatment period can also be normalized to the baseline SAF value prior to treatment to arrive at the percentage change relative to baseline (e.g., ΔSAF_{Baseline}=ΔSAF/SAF_{Baseline}). Likewise, ΔΔSAF for a treatment period can also be normalized to the baseline SAF value prior to treatment to arrive at percentage change relative to the control population and the baseline (e.g., ΔΔSAF_{Baseline}=ΔΔSAF/SAF_{Baseline}).

### EXAMPLES

### Example 1

### Exemplary Cosmetic Compositions

Table 1 provides examples of cosmetic compositions suitable for use with the methods herein. The compositions are made by blending the A phase components with a suitable mixer (e.g., Tekmar RW20DZM) and heating to a temperature of 70-80° C. and maintaining the temperature while stirring. Separately, blend the B phase components with a suitable mixer and heat to 70-75° C., maintaining temperature while mixing. Phase B is added to Phase A while mixing well to emulsify. The emulsion is then milled using a suitable mill (e.g., Tekmar T-25) for 5 minutes. When the emulsion is at 60° C., phase C is added while continuing to mix. At 40° C., the ingredients of phase D and E are added to the emulsion. The emulsion is then milled using a suitable mill (Tekmar T-25) for 5 minutes resulting in a uniform product.

TABLE 1

| | 1 | 2 | 3 | 4 | 5 | 6 |
|---|---|---|---|---|---|---|
| Component | % | | | | | |
| Phase A | | | | | | |
| water | qs | qs | qs | qs | qs | qs |
| glycerol | 5.00 | 7.00 | 3.00 | 10.00 | 5.00 | 15.00 |
| disodium EDTA | 0.10 | 0.05 | 0.10 | 0.10 | 0.05 | 0.10 |
| Phase B | | | | | | |
| Isopropyl Isostearate | 5.00 | 2.50 | 1.33 | 2.50 | 5.00 | 2.50 |
| Isohexadecane | 1.00 | 1.50 | 3.00 | 1.00 | 3.00 | 5.00 |
| Distearyldimonium | 0.00 | 0.50 | 1.00 | 1.50 | 0.00 | 1.50 |
| Chloride | | | | | | |
| Steareth-2 | 0.50 | 2.00 | 1.00 | 1.00 | 1.50 | 3.00 |
| cetyl alcohol | 0.25 | 0.50 | 0.32 | 0.50 | 1.00 | 0.40 |
| tocopherol acetate | 0.00 | 0.50 | 0.50 | 0.50 | 0.25 | 1.00 |
| Steareth-21 | 0.50 | 1.00 | 0.40 | 0.80 | 1.25 | 2.00 |
| stearyl alcohol | 0.70 | 1.50 | 2.00 | 2.25 | 3.00 | 4.50 |
| behenyl alcohol | 0.80 | 1.00 | 0.40 | 0.60 | 1.50 | 0.60 |
| ethyl paraben | 0.20 | 0.20 | 0.20 | 0.20 | 0.20 | 0.20 |
| propyl paraben | 0.10 | 0.10 | 0.10 | 0.10 | 0.10 | 0.10 |
| polymethylsilsesquioxane | 1.25 | 2.50 | 2.00 | 0.50 | 0.25 | 1.50 |
| Phase C | | | | | | |
| Polyethylene | 1.50 | 1.00 | 1.50 | 2.00 | 1.25 | 1.00 |
| Phase D | | | | | | |
| Water | 5.00 | 10.00 | 10.00 | 5.00 | 10.00 | 15.00 |
| Nicotinamide Riboside | 2.00 | 5.00 | 5.00 | 2.50 | 4.00 | 7.00 |
| Chloride¹ (% w/v) | | | | | | |
| dexpanthenol | 0.25 | 0.50 | 0.50 | 2.00 | 1.00 | 2.00 |
| Phase E | | | | | | |
| benzyl alcohol | 0.25 | 0.25 | 0.25 | 0.25 | 0.25 | 0.25 |
| dimethicone/dimethiconol | 0.5 | 1.00 | 2.00 | 0.25 | 2.00 | 2.00 |

¹available from Chromadex, Irvine CA

### Example 2

### Inhibiting Melanosome Uptake

This example compares the ability of nicotinamide riboside (“NR”) and niacinamide to inhibit melanosome uptake into keratinocytes as compared to a vehicle control (i.e., a composition identical to the test composition (NR) and positive control (niacinamide) except it does not include niacinamide or nicotinamide riboside).

Melanosome Uptake Assay

Melanosome uptake was determined as follows. Carboxyfluorescein diacetate (“CFDA”) (available from Sigma, St. Louis, Mo.) labeled melanosomes were prepared by incubating CFDA dye in SKMEL-188 culture cells (available from Sloan Kettering Institute) for 2 days at 37° C. in a THERMO SCIENTIFIC FORMA brand CO₂ incubator (available from Fisher Scientific, Waltham, Mass.). On day 3 of the test, melanosomes were isolated from SKMEL-188 cells by step density centrifugation with sucrose solutions layered with different densities, which is well known in the art. Melanosomes were taken from the 1.6 M-2.0 M sucrose layers. The isolated melanosomes were placed in each well of a 6-well plate along with human neonatal keratinocytes (available from Thermo) (approximately 50,000 keratinocytes/well). 2 ml of the appropriate medium (i.e., the test composition, the positive control or the vehicle control) was added to each of the wells to produce 3 test wells (i.e., 3 replicates of each of three composition tested). The test composition was made by adding nicotinamide riboside chloride powder (available from Chromadex, Irvine, Calif.) to EPILIFE brand keratinocyte medium to produce a solution of 0.0025 w/v % nicotinamide riboside. The positive control was made by adding niacinamide to EPILIFE brand keratinocyte medium to produce a solution of 0.0025 w/v % niacinamide. Unmodified keratinocyte medium was used as a vehicle control.

The resulting test plates were incubated for two days in EPILIFE brand keratinocyte medium. On day 6 of the test, the keratinocytes were detached from the plates using trypsin and fluorescent-label counted by flow cytometry using an LSRFortessa brand flow cytometer (available from Becton Dickinson, NJ). The percentage of cells that had fluorescence (from CFDA label) was used as a metric to determine melanosome uptake. Keratinocytes containing detectable levels of CFDA were counted as a fraction of all keratinocytes passing through the flow cytometer (indicated as % uptake in Table 2). A higher percentage corresponds to a higher level of melanosome uptake into the keratinocytes.

Table 2 illustrates the results of the test. As shown in Table 2, the positive control appears to inhibit the rate of melanosome uptake compared to the vehicle control, which was expected. Surprisingly, the nicotinamide riboside appears to increase the rate of melanosome uptake compared to the vehicle control, which was not expected and which might initially suggest that: 1) nicotinamide riboside could worsen the appearance of pigmented spots; and/or 2) nicotinamide riboside, while being an analogue of niacinamide, does not have all the same mechanisms of action as niacinamide, especially with regard to inhibiting melanosome uptake by keratinocytes.

TABLE 2

| | % | Rate of Melanosome Uptake |
|---|---|---|
| Sample | Uptake | Versus Control |
| Vehicle Control | 36 | 100% |
| Positive Control (0.0025 w/v % | 26 | 72% |
| niacinamide) | | |
| Test Composition (0.0025 | 67 | 186% |
| w/v % NR) | | |

### Example 3

### In Vitro UV Stress Test

This example compares the ability of nicotinamide riboside and niacinamide to reduce the amount of HMGB1 protein released from keratinocytes subjected to stress from ultraviolet (“UV”) radiation.

Human neonatal keratinocytes (available from Thermo) were placed in each well of four 12-well plates. Each well also contained 2 ml of EPILIFE brand keratinocyte medium. The plates were incubated at 37° C. in a CO₂ incubator until cell confluency reached 70%. At this point, the cells, except for the negative control, were exposed to 15 mJ/cm² UVB (i.e., UV radiation with a wavelength of from 315-280 nm) in a BIO-SUN brand UV irradiating system (available from Vilber Lourmat, France). After UVB exposure, the keratinocyte medium in each well was replaced with an appropriate medium (i.e., niacinamide medium, nicotinamide riboside medium or a control medium) to produce the test plates. The test plates were incubated for 24 hours at 37° C. in a CO₂ incubator, after which the medium in each cell was removed and the HMGB1 level measured using a conventional HMGB1 ELISA kit (REF # ST51011, available from IBL International, Canada) according to the manufacturer's instructions.

The test media were made by adding either niacinamide or nicotinamide riboside to EPILIFE brand keratinocyte medium to produce a 0.001 w/v % solution. The control medium was unmodified keratinocyte medium.

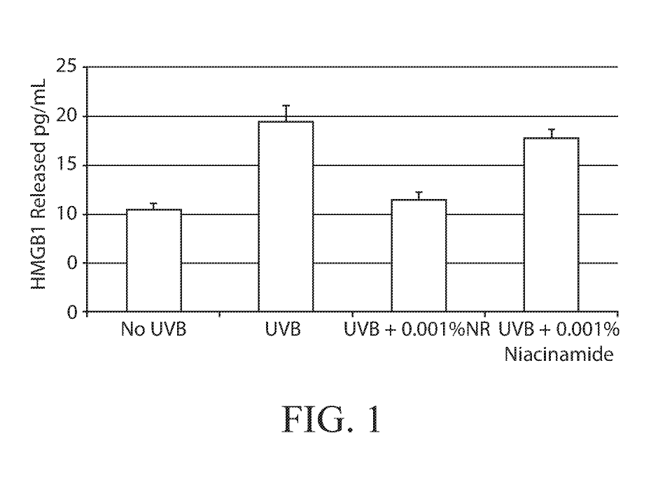

Table 3 and FIG. 1 illustrate the results of the test. As shown in Table 3 and FIG. 1, the untreated keratinocytes exposed to UVB radiation released more HMGB1 protein than the untreated cells that were not exposed to UVB, which is expected. Treating keratinocytes with niacinamide appears to have had no significant effect on the amount of HMGB1 protein released by keratinocytes exposed to UVB radiation when compared to the untreated UVB exposed cells. Surprisingly, the UVB exposed, nicotinamide riboside treated keratinocytes released less HMGB1 protein than the untreated, UVB-exposed keratinocytes. The p-values shown in Table 3 are student's T-test, 2-sided, equal variance. P-values of less than 0.05 are considered statistically significant.

TABLE 3

| | HMGB1 Released | p-value (vs. UVB |
|---|---|---|
| Treatment | (pg/ml) | exposed, untreated cells) |
| No UV exposure, | 10.5 | <0.05 |
| untreated (negative | | |
| control) | | |
| UV exposure, untreated | 19.4 | 1 |
| (positive control) | | |
| UV exposure + 0.001% | 11.5 | <0.05 |
| Nicotinamide Riboside | | |
| Chloride | | |
| UV exposure + 0.001% | 17.8 | 0.24 |
| Niacinamide | | |

### Example 4

### Clinical Study

This example demonstrates the ability of a cosmetic composition comprising an effective amount of nicotinamide riboside to improve the appearance of a hyperpigmented spot and lighten skin, relative to a baseline value or a control. Composition #3 from Table 1 was tested in this study. Approximately four months passed from the time the composition was made to the time it was tested. During this time, the composition was stored at about room temperature (i.e., ˜25 C).

The clinical study in this example is a 9-week, randomized, double-blinded, split-face, round robin study, which includes a 1 week normalization period and an 8 week test product usage period. The cosmetic compositions tested in the clinical study included a test composition comprising 5% nicotinamide riboside and the control composition set forth in Table 5. The control composition is an oil-in-water emulsion similar to conventional moisturizing lotions/creams and was made using conventional methods known in the art for making such compositions.

TABLE 4
Control Composition

| Component | % |
|---|---|
| Phase A | |
| water | qs |
| glycerol | 3.00 |
| disodium EDTA | 0.10 |
| Phase B | |
| Isopropyl Isostearate | 1.33 |
| Isohexadecane | 3.00 |
| cetearyl glucoside | 0.20 |
| cetyl alcohol | 0.32 |
| tocopherol acetate | 0.50 |
| PEG-100 stearate | 0.10 |
| stearyl alcohol | 0.48 |
| behenyl alcohol | 0.40 |
| ethyl paraben | 0.20 |
| propyl paraben | 0.10 |
| polymethylsilsesquioxane | 0.25 |
| Phase C | |
| polyacrylamide/C13-14 | 2.00 |
| isoparaffin/laureth-7 | |
| Phase D | |
| benzyl alcohol | 0.25 |
| dimethicone/dimethiconol | 2.00 |

Asian females aged 25 to 55 years old and having relative dark skin tone (L*<60, by Chromameter CR400) and a suitable number of hyperpigmented spots were selected to participate in the study. Prior to application of a test or control composition, the test subjects washed their face with OLAY DEEP PURIFY CLEANSER brand facial cleanser. In this example, after washing, the test product (i.e., composition #3 from Table 1) was applied to one side of the test subject's face, and the vehicle control was applied to the other side of the subject's face. This was done twice a day (morning/evening) during the test period. Dosage was 0.5 g per split face (forehead to jawline 4 mg/cm²). Measurements were taken at the start of the test period (baseline) and after 2, 4 and 8 weeks of treatment. Digital images were captured and analyzed for changes in L* and SAF according the Imaging Method described above. The data were statistically analyzed with a known Mixed Model (e.g., available from SAS Institute, Cary, N.C., U.S.A.) for repeated measures with the subject effect fitted as random, and the other effects (treatment, side (left and right), week, treatment-by-week interaction, age, baseline) fitted as fixed. Values are considered statistically significant if the p-value is less than or equal to 0.05.

The results of the clinical study are illustrated in Tables 5 and 6. Table 5 shows the change in lightness values (ΔL*) for the test composition relative to the control and baseline values at weeks 2, 4, and 8 for the test composition and the vehicle control. Baseline values for all test subjects were measured on Day 0 and averaged to provide a common baseline for use in the test. As shown in Tables 5, treatment with the test composition lightened the skin at week 8 (positive ΔL* value) relative to the baseline value, and at week 2, 4 and 8 relative to the control.

TABLE 5

| | | | | ΔL* vs. | | ΔL* | |
|---|---|---|---|---|---|---|---|
| | | Com- | L* | Base- | | vs. | |
| | N | position | Value | line | p-value | Control | p-value |
| Base- | | — | 57.326 | — | — | — | — |
| line | | | | | | | |
| Week 2 | 41 | Vehicle | 56.899 | −0.427 | <0.0001 | — | — |
| Week 4 | 41 | Control | 56.679 | −0.648 | <0.0001 | — | — |
| Week 8 | 41 | | 57.000 | −0.327 | <0.0001 | — | — |
| Week 2 | 41 | Test | 57.327 | 0.001 | 0.9986 | 0.428 | <0.0001 |
| Week 4 | 41 | Compo- | 57.368 | 0.041 | 0.6788 | 0.689 | <0.0001 |
| | | sition | | | | | |
| Week 8 | 41 | (5% NR) | 57.817 | 0.491 | <0.0001 | 0.817 | <0.0001 |

Table 6 shows the changes in SAF observed at weeks 2, 4, and 8 for the test composition and the vehicle control. As shown in Table 6, treatment with the test composition consistently reduced Spot Area Fraction at weeks 2, 4 and 8 relative to the baseline and the control composition. In contrast, the vehicle control does not appear to provide any significant reduction in SAF. The ΔSAF was determined by subtracting the baseline SAF from the SAF measured at each time point. The ΔΔSAF was determined by subtracting the ΔSAF of the control composition from the ΔSAF of the test composition at each time point. The % changes in SAF are also provided in Table 6.

TABLE 6

| | | | | | | | | % Change | % Change |
|---|---|---|---|---|---|---|---|---|---|
| | | | | | | | | ΔSAF | ΔΔSAF |
| | | | | ΔSAF | | | | normalized | normalized |
| | | | SAF | (vs. | | ΔΔSAF | | to Baseline | to Baseline |
| | N | Composition | (%) | baseline) | p-value | (vs. control) | p-value | SAF | SAF |
| Baseline | | — | 6.755 | — | — | | | | |
| Week 2 | 41 | A (Control) | 6.830 | 0.075 | 0.4930 | — | — | 1.1% | |
| Week 4 | 41 | A (Control) | 6.578 | −0.176 | 0.1669 | — | — | −2.6% | |
| Week 8 | 41 | A (Control) | 6.665 | −0.090 | 0.4757 | — | — | −1.3% | |
| Week 2 | 41 | C (5% NR) | 6.234 | −0.520 | <0.0001 | −0.596 | <0.0001 | −7.7% | −8.8% |
| Week 4 | 41 | C (5% NR) | 5.820 | −0.935 | <0.0001 | −0.758 | <0.0001 | −13.8% | −11.2% |
| Week 8 | 41 | C (5% NR) | 5.579 | −1.176 | <0.0001 | −1.086 | <0.0001 | −17.4% | −16.1% |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic method of reducing the level of High-Mobility Group Protein B1 (HMGB1) released from keratinocytes, comprising:
a. identifying a target portion of skin where reduction of HMGB1 level is desired; and
b. topically applying a cosmetic composition to the target portion of skin during a treatment period, wherein the cosmetic composition comprises an effective amount of nicotinamide riboside, and the treatment period is sufficient to reduce the level of HMGB1 released by keratinocytes.

2. The method of claim 1, wherein the HMGB1 level is reduced by at least 10%.

3. The method of claim 1, wherein the target portion of skin comprises a skin pigmentation disorder.

4. The method of claim 1, wherein the cosmetic composition is formulated with between about 0.05% and about 10% nicotinamide riboside.

5. The method of claim 4, wherein the cosmetic composition is formulated with between about 0.5% and about 6% nicotinamide riboside.

6. The method of claim 1, wherein the treatment period is at least 2 weeks, preferably at least 4 weeks, and more preferably at least 8 weeks.

7. The method of claim 1, wherein the reduction in HMGB1 level results in reduced melanocyte dendricity in the target portion of skin.

8. The method of claim 1, wherein the cosmetic composition comprising a dermatologically acceptable carrier.

9. The method of claim 9, wherein the cosmetic composition includes an additional ingredient selected from skin tone agents and hydrophilic diluents.

10. The method of claim 10, wherein the composition further comprises niacinamide.

11. A method of lightening skin, comprising:
a. identifying a target portion of skin where skin lightening is desired; and
b. topically applying a cosmetic composition to the target portion of skin during a treatment period, wherein the cosmetic composition is formulated with about 0.005% to about 20% by weight nicotinamide riboside and the treatment period is sufficient to lighten the target skin portion.

12. The method of claim 11, wherein the skin lightening corresponds to a positive change in L* value.

13. The method of claim 11, wherein the positive change in L* value is at least 0.1.

14. The method of claim 13, wherein the cosmetic composition is an emulsion.

15. The method of claim 13, wherein the cosmetic composition comprises from about 2% to about 20% of a spherical particle powder.

16. A method of improving the appearance of a hyperpigmented spot, comprising:
a. identifying a target portion of skin that includes a hyperpigmented spot; and
b. topically applying a cosmetic composition to at least a portion of the hyperpigmented spot during a treatment period, wherein the cosmetic composition is formulated with about 0.005% to about 20% by weight nicotinamide riboside and the treatment period is sufficient to reduce the Spot Area Fraction (SAF) of the hyperpigmented spot.

17. The method of claim 16, wherein the reduction in SAF wherein the reduction in SAF is at least 2%.

18. The method of claim 16, wherein the cosmetic composition provides a positive change in L* value to the hyperpigmented spot.

19. The method of claim 18, wherein the positive change in L* value is at least 0.1.

20. The method of claim 16, wherein the cosmetic composition comprises at least one additional skin tone agent.
